(19) United States
(12) Patent Application Publication
(10) Pub. No.: US 2020/0222432 A1
(43) Pub. Date: Jul. 16, 2020

(54) TREATMENT OF NON-INFLAMMATORY LESIONS

(71) Applicant: ALMIRALL, LLC., Exton, PA (US)

(72) Inventors: David BERK, Newport Beach, CA (US); Alexandre KAOUKHOV, Newport Beach, CA (US); Vilma SNIUKIENE, Lake Hopatcong, NJ (US); Carsten SCHMITZ, Irvine, CA (US)

(21) Appl. No.: 16/632,121

(22) PCT Filed: Jul. 20, 2018
(86) PCT No.: PCT/IB2018/000938

§ 371 Date: Jan. 17, 2020

Related U.S. Application Data
(60) Provisional application No. 62/535,572, filed on Jul. 21, 2017.

(51) Int. Cl. A61K 31/65; A61K 9/00; A61P 17/10

(52) U.S. Cl. CPC A61K 31/65; A61P 17/10; A61K 9/0053

(57) ABSTRACT

Provided herein are methods of treating non-inflammatory lesions and symptoms thereof comprising administering to a subject in need thereof a therapeutically effective amount of (4S,4aS,5aR,12aS)-4-dimethylamino-3,10,12,12a-tetrahydroxy-7-[(methoxy(methyl)amino)-methyl]-1,11-dioxo-1,4,4a,5,5a,6,11,12a-octahydro-naphthacene-2-carboxylic acid amide or pharmaceutically acceptable salts thereof. In certain embodiments, a crystalline mono hydrochloride, mono mesylate, or mono sulfate salt of (4S,4aS,5aR,12aS)-4-(dimethylamino)-3,10,12,12a-tetrahydroxy-7-[(methoxy(methyl)amino)methyl]-1,11-dioxo-1,4,4a,5,5a,6,11,12a-octahydro-naphthacene-2-carboxylic acid amide is administered.

The present application claims the benefit of U.S. provisional application No. 62/535,572, filed Jul. 21, 2017, the entire disclosure of which is incorporated by reference herein.

### FIELD OF THE INVENTION

The instant disclosure relates to treatment of non-inflammatory lesions.

### BACKGROUND OF THE INVENTION

Acne vulgaris, also referred to as acne, is typically an inflammatory skin disorder with inflammatory lesions such as papules and pustules. Acne may also be present with non-inflammatory lesions, also known as open and closed comedones. Acne is a disorder resulting from hormones affecting the sebaceous glands, which leads to plugged pores and outbreaks of lesions, or pimples. Acne is the most common skin disease in the United States, affecting nearly 17 million people. Severe acne can lead to disfiguration and permanent scarring.

Acne is described as a disorder of the pilosebaceous units (PSUs). Found over most of the body, PSUs consist of sebaceous glands, which make an oily substance that normally empties onto the skin surface through the opening of the follicle, also called a pore. When the follicle is plugged, the mixture of oil and cells allows bacteria that normally live on the skin to grow in the plugged follicles. Depending on the amount of oxygen that can penetrate the follicles, the bacteria may produce chemicals and enzymes and attract white blood cells that cause inflammation. The plugged follicle breaks down, the sebum, shed skin cells and bacteria disseminate into the nearby tissues, leading to lesions (i.e., inflammatory lesions) or pimples. Inflammatory lesions are characterized by the presence of papules, pustules, and nodules (cysts) and are classified as papulopustular and/or nodular. A severity grade based on a lesion count approximation is assigned as mild, moderate, or severe (Dermet Skin disease Atlas). Unlike inflammatory lesions, non-inflammatory lesions consist of open and closed comedones and usually appear before inflammatory lesions. Closed comedones or whiteheads appear as white microcysts. The follicular opening is barely perceptible. Open comedones or blackheads have a dilated follicular orifice that contains a plug with a dark surface. The opening may be small or very large.

Tetracycline compounds, or tetracyclines, are known “broad spectrum” antibiotics and have been widely used for therapeutic purposes. Tetracyclines are bacteriostatic drugs and act by binding reversibly to the 30S subunit of the bacterial ribosome. This inhibits the addition of amino acids to the growing peptide resulting in inhibition of protein synthesis. Tetracyclines also exhibit anti-inflammatory and anti-collagenolytic properties which are not related to antibiotic activity (Cutis, 75(4 Suppl): 6-11, Apr. 2005).

For example, tetracyclines have been found to be highly effective pharmacologically against rickettsiae and several gram-positive and gram-negative bacteria, including lymphogranuloma venereum, inclusion conjunctivitis, and psittacosis. Examples of pharmaceutically active tetracycline analogue compositions may be found in U.S. Pat. Nos. 2,980,584; 2,990,331; 3,062,717; 3,165,531; 3,454,697; 3,557,280; 3,674,859; 3,957,980; 4,018,889; 4,024,272; and 4,126,680. Some tetracyclines may also be used to treat inflammatory skin disorders, including dermatitis, psoriasis, pyoderma gangrenosum, acne, and rosacea.

After the widespread use of tetracyclines for both major and minor illnesses and diseases led to resistance to these antibiotics, substituted tetracycline compounds were developed to treat bacterial infections, inflammation, neoplasms, and other conditions. Examples of these tetracycline compounds include without limitation: chlortetracycline, doxycycline, minocycline, oxytetracycline, demeclocycline, methacycline, sancycline, chelocardin, rolitetracycline, lymecycline, apicycline, clomocycline, guamecycline, meglucycline, mepylcycline, penimepicycline, pipacycline, etamocycline, and penimocycline. For example, substituted tetracycline compounds have been disclosed in WO 2008/079339 and WO 2008/079363.

There remains a need for treatments of acne vulgaris, in particular, treatment of non-inflammatory lesions and symptoms thereof.

### SUMMARY OF THE INVENTION

One aspect of the present invention relates to a method of treatment of non-inflammatory lesions. Specifically, in this aspect, the treatment comprises administration to a subject in need of such treatment (4S,4aS,5aR,12aS)-4-dimethylamino-3,10,12,12a-tetrahydroxy-7-[(methoxy(methyl)amino)-methyl]-1,11-dioxo-1,4,4a,5,5a,6,11,12a-octahydro-naphthacene-2-carboxylic acid amide or a pharmaceutically acceptable salt thereof. One such salt may be a crystalline salt of (4S,4aS,5aR,12aS)-4-(dimethylamino)-3,10,12,12a-tetrahydroxy-7-[(methoxy(methyl)amino)methyl]-1,11-dioxo-1,4,4a,5,5a,6,11,12a-octahydro-naphthacene-2-carboxylic acid amide hydrochloride. This crystalline salt may be, for example, a mono hydrochloride, a mono mesylate, or a mono sulfate. Specifically, the crystalline salt may be (4S,4aS,5aR,12aS)-4-(dimethylamino)-3,10,12,12a-tetrahydroxy-7-[(methoxy(methyl)amino)methyl]-1,11-dioxo-1,4,4a,5,5a,6,11,12a-octahydro-naphthacene-2-carboxamide hydrochloride.

Another aspect of the present invention relates to (4S,4aS,5aR,12aS)-4-dimethylamino-3,10,12,12a-tetrahydroxy-7-[(methoxy(methyl)amino)-methyl]-1,11-dioxo-1,4,4a,5,5a,6,11,12a-octahydro-naphthacene-2-carboxylic acid amide or a pharmaceutically acceptable salt thereof for use in treating non-inflammatory lesions or for use in preparing a medicament for the treatment of non-inflammatory lesions.

The following description sets forth exemplary methods, parameters, and the like. It should be recognized, however, that such description is not intended as a limitation on the scope of the present disclosure but is instead provided as a description of exemplary embodiments.

### BRIEF DESCRIPTION OF THE DRAWINGS

FIG. 1 shows the mean absolute reduction in non-inflammatory lesions for subjects having 30 or more non-inflammatory lesions at baseline as described in Example 1.

FIG. 2 shows the mean percent reduction in non-inflammatory lesions for subjects having 30 or more non-inflammatory lesions at baseline as described in Example 1.

FIG. 3 shows the mean absolute reduction in non-inflammatory lesions for subjects having 30 or more non-inflammatory lesions at baseline as described in Example 2.

FIG. 4 shows the mean percent reduction in non-inflammatory lesions for subjects having 30 or more non-inflammatory lesions at baseline as described in Example 2.

FIG. 5 shows the mean absolute reduction and the percent change in non-inflammatory lesions from baseline to final visit (week 12) as described in the Reference Example.

### DETAILED DESCRIPTION OF THE INVENTION

It has now been found that non-inflammatory lesions and/or symptoms thereof, particularly those associated with acne vulgaris or facial acne vulgaris, can be treated using (4S,4aS,5aR,12aS)-4-dimethylamino-3,10,12,12a-tetrahydroxy-7-[(methoxy(methyl)amino)-methyl]-1,11-dioxo-1,4,4a,5,5a,6,11,12a-octahydro-naphthacene-2-carboxylic acid amide (sarecycline) or a pharmaceutically acceptable salt thereof. It has also been found that prophylactic treatment for non-inflammatory lesions and/or symptoms thereof can be performed using (4S,4aS,5aR,12a5)-4-dimethylamino-3,10,12,12a-tetrahydroxy-7-[(methoxy(methyl)amino)-methyl]-1,11-dioxo-1,4,4a,5,5a,6,11,12a-octahydro-naphthacene-2-carboxylic acid amide (sarecycline) or a pharmaceutically acceptable salt thereof.

Non-inflammatory lesions comprise open and closed comedones. An open comedo, commonly referred to as a blackhead, is a non-infected plugged hair follicle with a dilated or open orifice. A closed comedo, commonly referred to as a whitehead, is a non-infected plugged hair follicle having dilated follicular orifice that contains a plug with a dark skin surface.

Symptoms or signs of non-inflammatory lesions vary depending on the severity of the condition of the subject and may be associated with small epidermal cyst-like masses that manifest on the surface of the skin that may have a flesh-like color and/or dark spots, which may appear to be a brown or black color on the surface of the skin.

In one aspect, the present invention provides a method of treating non-inflammatory lesions and/or symptoms thereof comprising administering to a subject in need thereof (4S,4aS,5aR,12a5)-4-dimethylamino-3,10,12,12a-tetrahydroxy-7-[(methoxy(methyl)amino)-methyl]-1,11-dioxo-1,4,4a,5,5a,6,11,12a-octahydro-naphthacene-2-carboxylic acid amide or its pharmaceutically acceptable salt. In another aspect, the present invention provides a method of prophylactic treatment for non-inflammatory lesions and/or symptoms thereof comprising administering to a subject in need thereof (4S,4aS,5aR,12aS)-4-dimethylamino-3,10,12,12a-tetrahydroxy-7-[(methoxy(methyl)amino)-methyl]-1,11-dioxo-1,4,4a,5,5a,6,11,12a-octahydro-naphthacene-2-carboxylic acid amide or its pharmaceutically acceptable salt. A therapeutically effective amount of the drug, or a safe and clinically effective amount of the drug, is preferably administered to the subject, and, preferably, such administration is performed for a time period sufficient to reduce the number, size and/or occurrence of non-inflammatory lesions.

The term “therapeutically effective amount,” as used herein, refers to an amount that may be effective to elicit the desired biological or medical response, including the amount of a compound that, when administered to a subject for treating a condition, is sufficient to affect such treatment for the condition. The effective amount will vary depending on the condition and its severity, as well as the age, weight and physical condition of the subject, as well as the duration of treatment and the like.

The term “safe and clinically effective amount,” as used herein, refers to an amount of a compound or composition high enough to significantly positively modify the symptoms and/or condition to be treated, but low enough to avoid serious side effects (at a reasonable risk/benefit ratio), within the scope of sound medical judgment. The safe and clinically effective amount of active ingredient for use in the method of the invention herein will vary with the particular condition being treated, the age and physical condition of the patient to be treated, the severity of the condition, the duration of the treatment, the nature of concurrent therapy, the particular active ingredient being employed, the particular pharmaceutically-acceptable excipients utilized, and like factors within the knowledge and expertise of the attending physician.

The term “subject(s),” as used herein, refers to those to be treated in accordance with the present invention. Subjects who have and/or exhibit non-inflammatory lesions or symptoms thereof can be treated. Subjects who may be prone to developing non-inflammatory lesions or symptoms thereof can be treated. In certain embodiments, the subject may have at least 10 non-inflammatory lesions, at least 20 non-inflammatory lesions, at least 30 non-inflammatory lesions, at least 40 non-inflammatory lesions, at least 50 non-inflammatory lesions, at least 60 non-inflammatory lesions, at least 70 non-inflammatory lesions, at least 80 non-inflammatory lesions, at least 90 non-inflammatory lesions, at least 100 non-inflammatory lesions, or at least 110 non-inflammatory lesions. In some embodiments, the subject may have fewer than 10 non-inflammatory lesions.

In one specific aspect, the subject in accordance with the present invention is a human. In another aspect, the subject is a non-human mammal.

The terms “treatment” or “treating,” as used herein, include therapeutic and/or prophylactic treatment as described herein. With respect to the treatment of non-inflammatory lesions, such treatment includes the diminishment in the appearance, or size, or number of these non-inflammatory lesions on the skin and/or alleviation of at least one of the symptoms associated with non-inflammatory lesions.

The term “prophylactic treatment”, as used herein, includes treatment that reduces the increase in the size and/or number of non-inflammatory lesions on the skin that would otherwise occur without such treatment. Preferably, the prophylactic treatment is a preventative treatment of a subject, wherein the preventative treatment may prevent an increase in the number of non-inflammatory lesions. The subject in need of the prophylactic treatment may have no non-inflammatory lesions or may have any number of non-inflammatory lesions, such as those mentioned above. For example, prophylactic treatment may be appropriate for a subject who is prone to developing non-inflammatory lesions but who may not exhibit non-inflammatory lesions at time of treatment.

The term “pharmaceutically acceptable salt,” as used herein, refers to any adduct between two or more chemical species that are capable of undergoing proton transfer. As such, the term “pharmaceutically acceptable salt” encompasses adducts in which complete proton transfer has occurred, adducts in which partial proton transfer has occurred (e.g., in which an equilibrium mixture of charged and uncharged species is formed), and/or adducts in which proton transfer has not occurred but the chemical species are associated, e.g., by hydrogen bonding. It is understood that the term “pharmaceutically acceptable salt” also encompasses adducts in which close ion pairs are present. It will also be understood that the term “pharmaceutically acceptable salt” encompasses a continuum of adducts between those adducts in which complete proton transfer has occurred to form discrete ions and/or adducts in which two species are associated but proton transfer has not occurred or has only partially occurred. See, e.g., Childs et al. Mol. Pharmaceutics, 2007, 4 (3), pp 323-338. A given pharmaceutically acceptable salt can contain one or multiple adducts on this continuum.

Pharmaceutically acceptable salts include salts of acidic or basic groups. Pharmaceutically acceptable acid addition salts include, but are not limited to, hydrochloride, hydrobromide, hydroiodide, nitrate, mesylate, sulfate, bisulfate, phosphate, acid phosphate, isonicotinate, acetate, lactate, salicylate, citrate, tartrate, pantothenate, bitartrate, ascorbate, succinate, maleate gentisinate, fumarate, gluconate, glucaronate, saccharate, formate, benzoate, glutamate, methanesulfonate, ethanesulfonate, benzensulfonate, ptoluenesulfonate and pamoate (i.e., 1,1′-methylene-bis-(2-hydroxy-3-naphthoate)) salts. Suitable base salts include, but are not limited to, aluminum, calcium, lithium, magnesium, potassium, sodium, zinc, and diethanolamine salts.

In some embodiments, the pharmaceutically acceptable salt may be a crystalline salt of (4S,4aS,5aR,12a5)-4-dimethylamino-3,10,12,12a-tetrahydroxy-7-[(methoxy(methyl)amino)-methyl]-1,11-dioxo-1,4,4a,5,5a,6,11,12a-octahydro-naphthacene-2-carboxylic acid amide. Such crystalline salt may be selected from the group consisting of mono hydrochloride, mono mesylate, and mono sulfate. These crystalline salts are described in U.S. Pat. No. 9,255,068, and the contents of this patent are incorporated herein by reference in their entirety.

In one embodiment, (4S,4aS,5aR,12aS)-4-(dimethylamino)-3,10,12,12a-tetrahydroxy-7-[(methoxy(methyl)amino)methyl]-1,11-dioxo-1,4,4a,5, 5 a,6, 11,12a-octahydro-naphthacene-2-carboxamide hydrochloride having the structure represented by the following Formula I may be administered to a subject in need of the treatment:

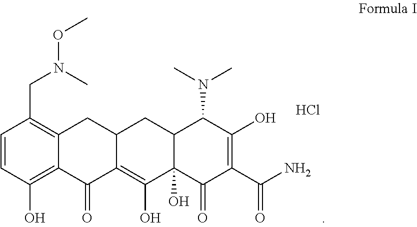 [SMILES: CON(C)Cc1ccc(O)c2c1CC1CC3[C@H](N(C)C)C(O)=C(C(N)=O)C(=O)[C@@]3(O)C(O)=C1C2=O ; Cl]

The therapeutically effective amount of (4S,4aS,5aR,12aS)-4-dimethylamino-3,10,12,12a-tetrahydroxy-7-[(methoxy(methyl)amino)-methyl]-1,11-dioxo-1,4,4a,5,5a,6,11,12a-octahydro-naphthacene-2-carboxylic acid amide or a pharmaceutically acceptable salt thereof will typically range from about 0.75 mg/kg per day to about 3.0 mg/kg per day. This amount may be from each of about 0.75 mg/kg and about 1.1 mg/kg to each of about 1.5 mg/kg per day and about 1.8 mg/kg per day. For example, the amount may be about 3.0 mg/kg per day or about 1.5 mg/kg per day.

In one embodiment, the therapeutically effective amount of (4S,4aS,5aR,12aS)-4-dimethylamino-3,10,12,12a-tetrahydroxy-7-[(methoxy(methyl)amino)-methyl]-1,11-dioxo-1,4,4a,5,5a,6,11,12a-octahydro-naphthacene-2-carboxylic acid amide or a pharmaceutically acceptable salt thereof employed is from about 1 mg to about 200 mg. This amount may be, for example, from each of about 60 mg and about 65 mg to each of about 100 mg, about 105 mg, about 150 mg, about 155 mg, and about 160 mg. Individual doses of (4S,4aS,5aR,12aS)-4-dimethylamino-3,10,12,12a-tetrahydroxy-7-[(methoxy(methyl)amino)-methyl]-1,11-dioxo-1,4,4a,5,5a,6,11,12a-octahydro-naphthacene-2-carboxylic acid amide or its pharmaceutically acceptable salt may be administered in a dose amount (via a single or multiple dosage forms) of about 60 mg, about 65 mg, about 100 mg, about 105 mg, about 150 mg, about 155 mg, and about 160 mg.

In certain embodiments, (4S,4aS,5aR,12aS)-4-dimethylamino-3,10,12,12a-tetrahydroxy-7-[(methoxy(methyl)amino)-methyl]-1,11-dioxo-1,4,4a,5,5a,6,11,12a-octahydro-naphthacene-2-carboxylic acid amide or a pharmaceutically acceptable salt thereof, preferably in its therapeutically effective amount, is administered at least once weekly, bi-weekly, or daily. In certain embodiments, such administration is performed for at least 1 week, at least 2 weeks, at least 3 weeks, at least 4 weeks, at least 5 weeks, at least 6 weeks, at least 7 weeks, at least 8 weeks, at least 9 weeks, at least 10 weeks, at least 11 weeks, or at least 12 weeks.

(4S,4aS,5aR,12aS)-4-dimethylamino-3,10,12,12a-tetrahydroxy-7-[(methoxy(methyl)amino)-methyl]-1,11-dioxo-1,4,4a,5,5a,6,11,12a-octahydro-naphthacene-2-carboxylic acid amide or a pharmaceutically acceptable salt thereof may be administered to the subject in need thereof at approximately the same time each day. For example, administration may occur in the evening. Administration may take place at least 1 hour prior to, or 2 hours after, a meal, such as an evening meal.

Preferably, (4S,4aS,5aR,12aS)-4-dimethylamino-3,10,12,12a-tetrahydroxy-7-[(methoxy(methyl)amino)-methyl]-1,11-dioxo-1,4,4a,5,5a,6,11,12a-octahydro-naphthacene-2-carboxylic acid amide or a pharmaceutically acceptable salt thereof as discussed throughout this disclosure is administered to the subject orally via an oral dosage form.

The term “oral dose” or “oral dosage form,” as used herein, refers to any pharmaceutical composition intended to be administered to the gastrointestinal tract of a subject via the mouth of said subject, and for purposes of the present disclosure, the oral dosage form as administered and/or delivered can be in liquid, solid, semi-solid, gelatinous form, or the like.

For instance, a solid oral dosage form may be a tablet, capsule, granule or the like. Such oral dosage forms may contain various excipients such as microcrystalline cellulose, sodium citrate, calcium carbonate, dicalcium phosphate and glycine, along with various disintegrants such as starch (and preferably corn, potato or tapioca starch), alginic acid and certain complex silicates, together with granulation binders like polyvinylpyrrolidone, sucrose, gelatin and acacia. In a certain embodiment, the oral dosage form may be film coated. Additionally, lubricating agents such as magnesium stearate, sodium lauryl sulfate and talc may be used. Other solid compositions may also be employed as fillers in the oral dosage form including lactose or milk sugar, as well as high molecular weight polyethylene glycols.

When aqueous suspensions and/or elixirs are desired for oral administration, they may include various sweetening or flavoring agents, coloring matter or dyes, and, if so desired, emulsifying and/or suspending agents, together with such diluents as water, ethanol, propylene glycol, glycerin and various like combinations thereof.

In one embodiment, administering a therapeutically effective amount of (4S,4aS,5aR,12aS)-4-dimethylamino-3,10,12,12a-tetrahydroxy-7-[(methoxy(methyl)amino)-methyl]-1,11-dioxo-1,4,4a,5,5a,6,11,12a-octahydro-naphthacene-2-carboxylic acid amide or a pharmaceutically acceptable salt may result in the mean percent reduction in non-inflammatory lesions from about 2% to about 75%. In another embodiment, such administration may result in the mean percent reduction in non-inflammatory lesions from about 4% to about 53%. In yet another embodiment, such administration may result in the mean percent reduction in non-inflammatory lesions from about 14% to about 43%. In another embodiment, such administration can result in the mean percent reduction in non-inflammatory lesions from about 16% to about 41%. In another embodiment, such administration can result in the mean percent reduction in non-inflammatory lesions from about 17% to about 39%. In another embodiment, such administration can result in the mean percent reduction in non-inflammatory lesions from about 20% to about 39%. In some embodiments, mean percent reduction of non-inflammatory lesions after about 3 weeks of treatment with a therapeutically effective amount of (4S,4aS,5aR,12aS)-4-dimethylamino-3,10,12,12a-tetrahydroxy-7-[(methoxy(methyl)amino)-methyl]-1,11-dioxo-1,4,4a,5,5a,6,11,12a-octahydro-naphthacene-2-carboxylic acid amide or a pharmaceutically acceptable salt may range from about 10% to about 25%. In some embodiments, mean percent reduction of non-inflammatory lesions after about 3 weeks of treatment with a therapeutically effective amount of (4S,4aS,5aR,12aS)-4-dimethylamino-3,10,12,12a-tetrahydroxy-7-[(methoxy(methyl)amino)-methyl]-1,11-dioxo-1,4,4a,5,5a,6,11,12a-octahydro-naphthacene-2-carboxylic acid amide or a pharmaceutically acceptable salt can range from about 16% to about 23%. In some embodiments, mean percent reduction of non-inflammatory lesions after about 6 weeks of treatment with a therapeutically effective amount of (4S,4aS,5aR,12aS)-4-dimethylamino-3,10,12,12a-tetrahydroxy-7-[(methoxy(methyl)amino)-methyl]-1,11-dioxo-1,4,4a,5,5a,6,11,12a-octahydro-naphthacene-2-carboxylic acid amide or a pharmaceutically acceptable salt may range from about 20% to about 35%. In some embodiments, mean percent reduction of non-inflammatory lesions after about 6 weeks of treatment with a therapeutically effective amount of (4S,4aS,5aR,12aS)-4-dimethylamino-3,10,12,12a-tetrahydroxy-7-[(methoxy(methyl)amino)-methyl]-1,11-dioxo-1,4,4a,5,5a,6,11,12a-octahydro-naphthacene-2-carboxylic acid amide or a pharmaceutically acceptable salt can range from about 25% to about 31%. In some embodiments, mean percent reduction of non-inflammatory lesions after about 9 weeks of treatment with a therapeutically effective amount of (4S,4aS,5aR,12aS)-4-dimethylamino-3,10,12,12a-tetrahydroxy-7-[(methoxy(methyl)amino)-methyl]-1,11-dioxo-1,4,4a,5,5a,6,11,12a-octahydro-naphthacene-2-carboxylic acid amide or a pharmaceutically acceptable salt may range from about 30% to about 45%. In some embodiments, mean percent reduction of non-inflammatory lesions after about 9 weeks of treatment with a therapeutically effective amount of (4S,4aS,5aR,12aS)-4-dimethylamino-3,10,12,12a-tetrahydroxy-7-[(methoxy(methyl)amino)-methyl]-1,11-dioxo-1,4,4a,5,5a,6,11,12a-octahydro-naphthacene-2-carboxylic acid amide or a pharmaceutically acceptable salt can range from about 32% to about 38%. In some embodiments, mean percent reduction of non-inflammatory lesions after about 12 weeks of treatment with a therapeutically effective amount of (4S,4aS,5aR,12aS)-4-dimethylamino-3,10,12,12a-tetrahydroxy-7-[(methoxy(methyl)amino)-methyl]-1,11-dioxo-1,4,4a,5,5a,6,11,12a-octahydro-naphthacene-2-carboxylic acid amide or a pharmaceutically acceptable salt may range from about 35% to about 50%. In some embodiments, mean percent reduction of non-inflammatory lesions after about 12 weeks of treatment with a therapeutically effective amount of (4S,4aS,5aR,12aS)-4-dimethylamino-3,10,12,12a-tetrahydroxy-7-[(methoxy(methyl)amino)-methyl]-1,11-dioxo-1,4,4a,5,5a,6,11,12a-octahydro-naphthacene-2-carboxylic acid amide or a pharmaceutically acceptable salt can range from about 36% to about 41%.

The difference in mean percent reduction in non-inflammatory lesions of a subject who receives a therapeutically effective amount of (4S,4aS,5aR,12aS)-4-dimethylamino-3,10,12,12a-tetrahydroxy-7-[(methoxy(methyl)amino)-methyl]-1,11-dioxo-1,4,4a,5,5a,6,11,12a-octahydro-naphthacene-2-carboxylic acid amide or a pharmaceutically acceptable salt compared to a placebo may range from about 1% to about 40%. In another embodiment, such administration may result in a difference in mean percent reduction in non-inflammatory lesions from about 1% to about 21%. In yet another embodiment, such administration may result in a difference in mean percent reduction in non-inflammatory lesions from about 1% to about 19%. In yet another embodiment, such administration can result in a difference in mean percent reduction in non-inflammatory lesions from about 1% to about 16%. In some embodiments, the difference in mean percent reduction of non-inflammatory lesions after about 3 weeks of treatment with a therapeutically effective amount of (4S,4aS,5aR,12aS)-4-dimethylamino-3,10,12,12a-tetrahydroxy-7-[(methoxy(methyl)amino)-methyl]-1,11-dioxo-1,4,4a,5,5a,6,11,12a-octahydro-naphthacene-2-carboxylic acid amide or a pharmaceutically acceptable salt may range from about 1% to about 14%. In some embodiments, the difference in mean percent reduction of non-inflammatory lesions after about 3 weeks of treatment with a therapeutically effective amount of (4S,4aS,5aR,12aS)-4-dimethylamino-3,10,12,12a-tetrahydroxy-7-[(methoxy(methyl)amino)-methyl]-1,11-dioxo-1,4,4a,5,5a,6,11,12a-octahydro-naphthacene-2-carboxylic acid amide or a pharmaceutically acceptable salt may range from about 1% to about 10%. In some embodiments, the difference in mean percent reduction of non-inflammatory lesions after about 3 weeks of treatment with a therapeutically effective amount of (4S,4aS,5aR,12aS)-4-dimethylamino-3,10,12,12a-tetrahydroxy-7-[(methoxy(methyl)amino)-methyl]-1,11-dioxo-1,4,4a,5,5a,6,11,12a-octahydro-naphthacene-2-carboxylic acid amide or a pharmaceutically acceptable salt can range from about 1% to about 8%. In some embodiments, the difference in mean percent reduction of non-inflammatory lesions after about 6 weeks of treatment with a therapeutically effective amount of (4S,4aS,5aR,12aS)-4-dimethylamino-3,10,12,12a-tetrahydroxy-7-[(methoxy(methylamino)-methyl]-1,11-dioxo-1,4,4a,5,5a,6,11,12a-octahydro-naphthacene-2-carboxylic acid amide or a pharmaceutically acceptable salt may range from about 1% to about 17%.. In some embodiments, the difference in mean percent reduction of non-inflammatory lesions after about 6 weeks of treatment with a therapeutically effective amount of (4S,4aS,5aR,12aS)-4-dimethylamino-3,10,12,12a-tetrahydroxy-7-[(methoxy(methyl)amino)-methyl]-1,11-dioxo-1,4,4a,5,5a,6,11,12a-octahydro-naphthacene-2-carboxylic acid amide or a pharmaceutically acceptable salt may range from about 1% to about 12%. In some embodiments, the difference in mean percent reduction of non-inflammatory lesions after about 6 weeks of treatment with a therapeutically effective amount of (4S,4aS,5aR,12aS)-4-dimethylamino-3,10,12,12a-tetrahydroxy-7-[(methoxy(methyl)amino)-methyl]-1,11-dioxo-1,4,4a,5,5a,6,11,12a-octahydro-naphthacene-2-carboxylic acid amide or a pharmaceutically acceptable salt can range from about 7% to about 11%. In some embodiments, the difference in mean percent reduction of non-inflammatory lesions after about 9 weeks of treatment with a therapeutically effective amount of (4S,4aS,5aR,12aS)-4-dimethylamino-3,10,12,12a-tetrahydroxy-7-[(methoxy(methyl)amino)-methyl]-1,11-dioxo-1,4,4a,5,5a,6,11,12a-octahydro-naphthacene-2-carboxylic acid amide or a pharmaceutically acceptable salt may range from about 1% to about 19%. In some embodiments, the difference in mean percent reduction of non-inflammatory lesions after about 9 weeks of treatment with a therapeutically effective amount of (4S,4aS,5aR,12aS)-4-dimethylamino-3,10,12,12a-tetrahydroxy-7-[(methoxy(methyl)amino)-methyl]-1,11-dioxo-1,4,4a,5,5a,6,11,12a-octahydro-naphthacene-2-carboxylic acid amide or a pharmaceutically acceptable salt can range from about 1% to about 13%. In some embodiments, the difference in mean percent reduction of non-inflammatory lesions after about 9 weeks of treatment with a therapeutically effective amount of (4S,4aS,5aR,12aS)-4-dimethylamino-3,10,12,12a-tetrahydroxy-7-[(methoxy(methyl)amino)-methyl]-1,11-dioxo-1,4,4a,5,5a,6,11,12a-octahydro-naphthacene-2-carboxylic acid amide or a pharmaceutically acceptable salt can range from about 6% to about 10%. In some embodiments, the difference in mean percent reduction of non-inflammatory lesions after about 12 weeks of treatment with a therapeutically effective amount of (4S,4aS,5aR,12aS)-4-dimethylamino-3,10,12,12a-tetrahydroxy-7-[(methoxy(methyl)amino)-methyl]-1,11-dioxo-1,4,4a,5,5a,6,11,12a-octahydro-naphthacene-2-carboxylic acid amide or a pharmaceutically acceptable salt may range from about 1% to about 20%. In some embodiments, the difference in mean percent reduction of non-inflammatory lesions after about 12 weeks of treatment with a therapeutically effective amount of (4S,4aS,5aR,12aS)-4-dimethylamino-3,10,12,12a-tetrahydroxy-7-[(methoxy(methyl)amino)-methyl]-1,11-dioxo-1,4,4a,5,5a,6,11,12a-octahydro-naphthacene-2-carboxylic acid amide or a pharmaceutically acceptable salt can range from about 1% to about 15%. In some embodiments, the difference in mean percent reduction of non-inflammatory lesions after about 12 weeks of treatment with a therapeutically effective amount of (4S,4aS,5aR,12aS)-4-dimethylamino-3,10,12,12a-tetrahydroxy-7-[(methoxy(methyl)amino)-methyl]-1,11-dioxo-1,4,4a,5,5a,6,11,12a-octahydro-naphthacene-2-carboxylic acid amide or a pharmaceutically acceptable salt can range from about 5% to about 11%..

In one embodiment, administering a therapeutically effective amount of (4S,4aS,5aR,12aS)-4-dimethylamino-3,10,12,12a-tetrahydroxy-7-[(methoxy(methyl)amino)-methyl]-1,11-dioxo-1,4,4a,5,5a,6,11,12a-octahydro-naphthacene-2-carboxylic acid amide or a pharmaceutically acceptable salt may result in a mean absolute reduction in non-inflammatory lesions from about 1 lesion to about 40 lesions. In another embodiment, such administration may result in the mean absolute reduction in non-inflammatory lesions from about 1 lesions to about 29 lesions. In another embodiment, such administration may result in the mean absolute reduction in non-inflammatory lesions from about 5 lesions to about 23 lesions. In another embodiment, such administration can result in the mean absolute reduction in non-inflammatory lesions from about 9 lesions to about 20 lesions. In some embodiments, mean absolute reduction in non-inflammatory lesions after about 3 weeks of treatment with a therapeutically effective amount of (4S,4aS,5aR,12aS)-4-dimethylamino-3,10,12,12a-tetrahydroxy-7-[(methoxy(methyl)amino)-methyl]-1,11-dioxo-1,4,4a,5,5a,6,11,12a-octahydro-naphthacene-2-carboxylic acid amide or a pharmaceutically acceptable salt may range from about 1 lesion to about 20 lesions. In some embodiments, mean absolute reduction in non-inflammatory lesions after about 3 weeks of treatment with a therapeutically effective amount of (4S,4aS,5aR,12aS)-4-dimethylamino-3,10,12,12a-tetrahydroxy-7-[(methoxy(methyl)amino)-methyl]-1,11-dioxo-1,4,4a,5,5a,6,11,12a-octahydro-naphthacene-2-carboxylic acid amide or a pharmaceutically acceptable salt can range from about 8 lesions to about 12 lesions. Ins some embodiments, mean absolute reduction in non-inflammatory lesions after about 3 weeks of treatment with a therapeutically effective amount of (4S,4aS,5aR,12aS)-4-dimethylamino-3,10,12,12a-tetrahydroxy-7-[(methoxy(methyl)amino)-methyl]-1,11-dioxo-1,4,4a,5,5a,6,11,12a-octahydro-naphthacene-2-carboxylic acid amide or a pharmaceutically acceptable salt can range from about 9 lesions to about 11 lesions. In some embodiments, mean absolute reduction in non-inflammatory lesions after about 6 weeks of treatment with a therapeutically effective amount of (4S,4aS,5aR,12aS)-4-dimethylamino-3,10,12,12a-tetrahydroxy-7-[(methoxy(methyl)amino)-methyl]-1,11-dioxo-1,4,4a,5,5a,6,11,12a-octahydro-naphthacene-2-carboxylic acid amide or a pharmaceutically acceptable salt may range from about 4 lesions to about 24 lesions. In some embodiments, mean absolute reduction in non-inflammatory lesions after about 6 weeks of treatment with a therapeutically effective amount of (4S,4aS,5aR,12aS)-4-dimethylamino-3,10,12,12a-tetrahydroxy-7-[(methoxy(methyl)amino)-methyl]-1,11-dioxo-1,4,4a,5,5a,6,11,12a-octahydro-naphthacene-2-carboxylic acid amide or a pharmaceutically acceptable salt can range from about 12 lesions to about 15 lesions. In some embodiments, mean absolute reduction in non-inflammatory lesions after about 6 weeks of treatment with a therapeutically effective amount of (4S,4aS,5aR,12aS)-4-dimethylamino-3,10,12,12a-tetrahydroxy-7-[(methoxy(methyl)amino)-methyl]-1,11-dioxo-1,4,4a,5,5a,6,11,12a-octahydro-naphthacene-2-carboxylic acid amide or a pharmaceutically acceptable salt can range from about 13 lesions to about 14 lesions. In some embodiments, mean absolute reduction in non-inflammatory lesions after about 9 weeks of treatment with a therapeutically effective amount of (4S,4aS,5aR,12aS)-4-dimethylamino-3,10,12,12a-tetrahydroxy-7-[(methoxy(methyl)amino)-methyl]-1,11-dioxo-1,4,4a,5,5a,6,11,12a-octahydro-naphthacene-2-carboxylic acid amide or a pharmaceutically acceptable salt may range from about 6 lesions to about 29 lesions. In some embodiments, mean absolute reduction in non-inflammatory lesions after about 9 weeks of treatment with a therapeutically effective amount of (4S,4aS,5aR,12aS)-4-dimethylamino-3,10,12,12a-tetrahydroxy-7-[(methoxy(methyl)amino)-methyl]-1,11-dioxo-1,4,4a,5,5a,6,11,12a-octahydro-naphthacene-2-carboxylic acid amide or a pharmaceutically acceptable salt can range from about 15 lesions to about 20 lesions. In some embodiments, mean absolute reduction in non-inflammatory lesions after about 9 weeks of treatment with a therapeutically effective amount of (4S,4aS,5aR,12aS)-4-dimethylamino-3,10,12,12a-tetrahydroxy-7-[(methoxy(methyl)amino)-methyl]-1,11-dioxo-1,4,4a,5,5a,6,11,12a-octahydro-naphthacene-2-carboxylic acid amide or a pharmaceutically acceptable salt can range from about 16 lesions to about 18 lesions. In some embodiments, mean absolute reduction in non-inflammatory lesions after about 12 weeks of treatment with a therapeutically effective amount of (4S,4aS,5aR,12aS)-4-dimethylamino-3,10,12,12a-tetrahydroxy-7-[(methoxy(methyl)amino)-methyl]-1,11-dioxo-1,4,4a,5,5a,6,11,12a-octahydro-naphthacene-2-carboxylic acid amide or a pharmaceutically acceptable salt may range from about 8 lesions to about 29 lesions. In some embodiments, mean absolute reduction in non-inflammatory lesions after about 12 weeks of treatment with a therapeutically effective amount of (4S,4aS,5aR,12aS)-4-dimethylamino-3,10,12,12a-tetrahydroxy-7-[(methoxy(methyl)amino)-methyl]-1,11-dioxo-1,4,4a,5,5a,6,11,12a-octahydro-naphthacene-2-carboxylic acid amide or a pharmaceutically acceptable salt can range from about 17 lesions to about 21 lesions. In some embodiments, mean absolute reduction in non-inflammatory lesions after about 12 weeks of treatment with a therapeutically effective amount of (4S,4aS,5aR,12aS)-4-dimethylamino-3,10,12,12a-tetrahydroxy-7-[(methoxy(methyl)amino)-methyl]-1,11-dioxo-1,4,4a,5,5a,6,11,12a-octahydro-naphthacene-2-carboxylic acid amide or a pharmaceutically acceptable salt can range from about 18 lesions to about 20 lesions.

The difference in mean absolute reduction in non-inflammatory lesions of a subject who receives a therapeutically effective amount of (4S,4aS,5aR,12aS)-4-dimethylamino-3,10,12,12a-tetrahydroxy-7-[(methoxy(methyl)amino)-methyl]-1,11-dioxo-1,4,4a,5,5a,6,11,12a-octahydro-naphthacene-2-carboxylic acid amide or a pharmaceutically acceptable salt compared to a placebo may range from about 1 lesion to about 15 lesions. In another embodiment, such administration may result in a difference in mean absolute reduction in non-inflammatory lesions from about 1 lesion to about 10 lesions. In yet another embodiment, such administration may result in a difference in mean absolute reduction in non-inflammatory lesions from about 1 lesion to about 8 lesions. In some embodiments, the difference in mean absolute reduction of non-inflammatory lesions after about 3 weeks of treatment with a therapeutically effective amount of (4S,4aS,5aR,12aS)-4-dimethylamino-3,10,12,12a-tetrahydroxy-7-[(methoxy(methyl)amino)-methyl]-1,11-dioxo-1,4,4a,5,5a,6,11,12a-octahydro-naphthacene-2-carboxylic acid amide or a pharmaceutically acceptable salt may range from about 1 lesion to about 7 lesions. In some embodiments, the difference in mean absolute reduction of non-inflammatory lesions after about 3 weeks of treatment with a therapeutically effective amount of (4S,4aS,5aR,12aS)-4-dimethylamino-3,10,12,12a-tetrahydroxy-7-[(methoxy(methyl)amino)-methyl]-1,11-dioxo-1,4,4a,5,5a,6,11,12a-octahydro-naphthacene-2-carboxylic acid amide or a pharmaceutically acceptable salt can range from about 1 lesion to about 4 lesions. In some embodiments, the difference in mean absolute reduction of non-inflammatory lesions after about 3 weeks of treatment with a therapeutically effective amount of (4S,4aS,5aR,12aS)-4-dimethylamino-3,10,12,12a-tetrahydroxy-7-[(methoxy(methyl)amino)-methyl]-1,11-dioxo-1,4,4a,5,5a,6,11,12a-octahydro-naphthacene-2-carboxylic acid amide or a pharmaceutically acceptable salt can range from about 1 lesion to about 2 lesions. In some embodiments, the difference in mean absolute reduction of non-inflammatory lesions after about 6 weeks of treatment with a therapeutically effective amount of (4S,4aS,5aR,12aS)-4-dimethylamino-3,10,12,12a-tetrahydroxy-7-[(methoxy(methyl)amino)-methyl]-1,11-dioxo-1,4,4a,5,5a,6,11,12a-octahydro-naphthacene-2-carboxylic acid amide or a pharmaceutically acceptable salt may range from about 1 lesion to about 9 lesions. In some embodiments, the difference in mean absolute reduction of non-inflammatory lesions after about 6 weeks of treatment with a therapeutically effective amount of (4S,4aS,5aR,12aS)-4-dimethylamino-3,10,12,12a-tetrahydroxy-7-[(methoxy(methyl)amino)-methyl]-1,11-dioxo-1,4,4a,5,5a,6,11,12a-octahydro-naphthacene-2-carboxylic acid amide or a pharmaceutically acceptable salt can range from about 1 lesion to about 5 lesions. In some embodiments, the difference in mean absolute reduction of non-inflammatory lesions after about 6 weeks of treatment with a therapeutically effective amount of (4S,4aS,5aR,12aS)-4-dimethylamino-3,10,12,12a-tetrahydroxy-7-[(methoxy(methyl)amino)-methyl]-1,11-dioxo-1,4,4a,5,5a,6,11,12a-octahydro-naphthacene-2-carboxylic acid amide or a pharmaceutically acceptable salt can range from about 2 lesions to about 3 lesions. In some embodiments, the difference in mean absolute reduction of non-inflammatory lesions after about 9 weeks of treatment with a therapeutically effective amount of (4S,4aS,5aR,12aS)-4-dimethylamino-3,10,12,12a-tetrahydroxy-7-[(methoxy(methyl)amino)-methyl]-1,11-dioxo-1,4,4a,5,5a,6,11,12a-octahydro-naphthacene-2-carboxylic acid amide or a pharmaceutically acceptable salt may range from about 2 lesions to about 11 lesions. In some embodiments, the difference in mean absolute reduction of non-inflammatory lesions after about 9 weeks of treatment with a therapeutically effective amount of (4S,4aS,5aR,12aS)-4-dimethylamino-3,10,12,12a-tetrahydroxy-7-[(methoxy(methyl)amino)-methyl]-1,11-dioxo-1,4,4a,5,5a,6,11,12a-octahydro-naphthacene-2-carboxylic acid amide or a pharmaceutically acceptable salt can range from about 1 lesion to about 7 lesions. In some embodiments, the difference in mean absolute reduction of non-inflammatory lesions after about 9 weeks of treatment with a therapeutically effective amount of (4S,4aS,5aR,12aS)-4-dimethylamino-3,10,12,12a-tetrahydroxy-7-[(methoxy(methyl)amino)-methyl]-1,11-dioxo-1,4,4a,5,5a,6,11,12a-octahydro-naphthacene-2-carboxylic acid amide or a pharmaceutically acceptable salt can range from about 3 lesions to about 5 lesions. In some embodiments, the difference in mean absolute reduction of non-inflammatory lesions after about 12 weeks of treatment with a therapeutically effective amount of (4S,4aS,5aR,12aS)-4-dimethylamino-3,10,12,12a-tetrahydroxy-7-[(methoxy(methyl)amino)-methyl]-1,11-dioxo-1,4,4a,5,5a,6,11,12a-octahydro-naphthacene-2-carboxylic acid amide or a pharmaceutically acceptable salt may range from about 1 lesion to about 12 lesions. In some embodiments, the difference in mean absolute reduction of non-inflammatory lesions after about 12 weeks of treatment with a therapeutically effective amount of (4S,4aS,5aR,12aS)-4-dimethylamino-3,10,12,12a-tetrahydroxy-7-[(methoxy(methyl)amino)-methyl]-1,11-dioxo-1,4,4a,5,5a,6,11,12a-octahydro-naphthacene-2-carboxylic acid amide or a pharmaceutically acceptable salt can range from about 1 lesion to about 8 lesions. In some embodiments, the difference in mean absolute reduction of non-inflammatory lesions after about 12 weeks of treatment with a therapeutically effective amount of (4S,4aS,5aR,12aS)-4-dimethylamino-3,10,12,12a-tetrahydroxy-7-[(methoxy(methyl)amino)-methyl]-1,11-dioxo-1,4,4a,5,5a,6,11,12a-octahydro-naphthacene-2-carboxylic acid amide or a pharmaceutically acceptable salt can range from about 3 lesions to about 6 lesions.

Unless defined otherwise, all technical and scientific terms used herein have the same meaning as commonly understood by one of ordinary skill in the art to which this invention belongs. Although any methods and materials similar or equivalent to those described herein can also be used in the practice or testing of the described invention, the preferred methods and materials are now described. All publications mentioned herein are incorporated herein by reference to disclose and describe the methods and/or materials in connection with the publications cited.

Where a range of values is provided, it is understood that each intervening value, to the tenth of the unit of the lower limit unless the context clearly dictates otherwise, between the upper and lower limit of that range and any other stated or intervening value in that stated range is encompassed within the invention. The upper and lower limits of these smaller ranges which may independently be included in the smaller ranges also is encompassed within the invention, subject to any specifically excluded limit in the stated range. Where the stated range includes one or both of the limits, ranges excluding either both of those included limits also are included in the invention.

It must also be noted that as used herein and in the appended claims, the singular forms “a,” “and” and “the” include plural referents unless the context clearly dictates otherwise. All technical and scientific terms used herein have the same meaning.

The publications discussed herein are incorporated herein by reference in their entirety and are provided solely for their disclosure prior to the filing date of the present application. Nothing herein is to be construed as an admission that the described invention is not entitled to antedate such publication by virtue of prior invention. Further, the dates of publication provided may be different from the actual publication dates which may need to be independently confirmed.

While the described invention has been described with reference to the specific embodiments thereof it should be understood by those skilled in the art that various changes may be made and equivalents may be substituted without departing from the true spirit and scope of the invention. In addition, many modifications may be made to adopt a particular situation, material, composition of matter, process, process step or steps, to the objective spirit and scope of the described invention. All such modifications are intended to be within the scope of the claims appended hereto.

### EXAMPLES

The following examples are provided to further aid in understanding the embodiments disclosed in the application, and presuppose an understanding of conventional methods well known to those persons having ordinary skill in the art to which the examples pertain. The particular materials and conditions described hereunder are intended to exemplify particular aspects of embodiments disclosed herein and should not be construed to limit the reasonable scope thereof.

### Example 1

Approximately 1.5 mg/kg per day of the mono hydrochloride crystalline salt of (4S,4aS,5aR,12a5)-4-dimethylamino-3,10,12,12a-tetrahydroxy-7-[(methoxy(methyl)amino)-methyl]-1,11-dioxo-1,4,4a,5,5a,6,11,12a-octahydro-naphthacene-2-carboxylic acid amide (i.e., Formula I) was administered to 483 subjects, and a placebo was administered to 485 subjects. The subjects of this study included males and females 9 to 45 years of age with moderate to severe facial acne vulgaris and no disorders that would preclude the use of tetracycline-class antibiotics. Subjects were treated for a total of 12 weeks and returned to a clinic to be assessed on Weeks 3, 6, 9, and 12 of treatment. Subjects were assessed through lesion counts and Investigator's Global Assessment (“IGA”) scores.

Subjects having facial acne vulgaris with: 20 to 50 inflammatory lesions (papules, pustules, and nodules), up to 100 non-inflammatory lesions (open and closed comedones), no more than two nodules and an IGA score of moderate (3) or severe (4) were included in the study.

The following is the IGA Scale used to assess acne vulgaris:

| Score | Grade | Description |
|---|---|---|
| 0 | Clear | No evidence of papules or pustules |
| 1 | Almost | Rare: inflammatory papules (papules must be resolving |
| | Clear | and may be hyperpigmented, though not pink-red) |
| 2 | Mild | Few: inflammatory lesions (papules/pustules only; no |
| | | nodulocytic lesions) |
| 3 | Moderate | Multiple: inflammatory lesions present; |
| | | many papules/pustules; there may or |
| | | may not be a few nodulocytic lesions |
| 4* | Severe | Inflammatory lesions are more apparent, many |
| | | papules/pustules; there may or may not be a few |
| | | nodulocytic lesions |

*Acne that worsens beyond Grade 4 must be recorded as an adverse event on the case report form (CRF).

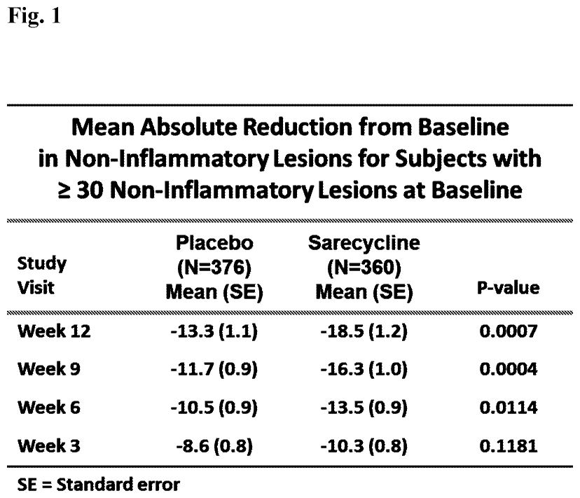

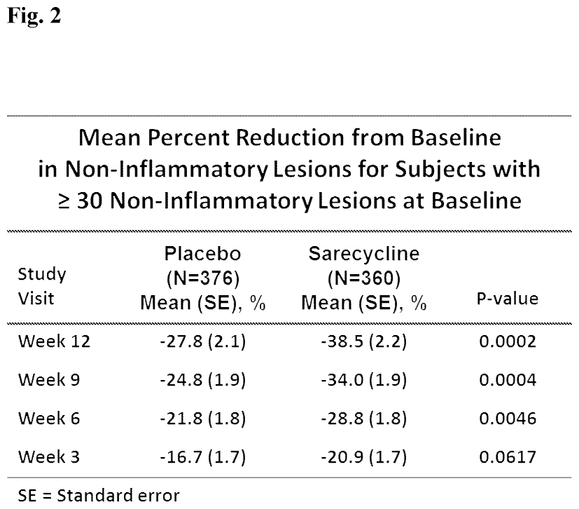

Data was analyzed for a subset of subjects having 30 or more non-inflammatory lesions. The effects of Formula I were evaluated for such subjects in comparison to a placebo. 360 subjects were given 1.5 mg/kg per day of Formula I, and 376 subjects were given a daily dose of a placebo. Each subject was instructed to take the daily dose at the same time each day at least 1 hour prior to or 2 hours after eating. FIG. 1 demonstrates the mean absolute reduction in non-inflammatory lesions for participants having 30 or more non-inflammatory lesions on the face at baseline who received daily doses of Formula I compared to the placebo. FIG. 2 demonstrates the mean percent reduction from baseline for subjects having 30 or more non-inflammatory lesions on the face at baseline. Results in these figures were based on a statistical (analysis of covariance) model used to estimate the mean treatment effects of Formula I and placebo, after adjusting for potential effects of investigative site and baseline value. In addition, the statistical superiority of Formula I relative to placebo is tested and p-values provided for each study visit.

### Example 2

Approximately 1.5 mg/kg per day of the mono hydrochloride crystalline salt of (4S,4aS,5aR,12aS)-4-dimethylamino-3,10,12,12a-tetrahydroxy-7-[(methoxy(methyl)amino)-methyl]-1,11-dioxo-1,4,4a,5,5a,6,11,12a-octahydro-naphthacene-2-carboxylic acid amide (i.e., Formula I) was administered to 519 subjects, and a placebo was administered 515 subjects. The subjects of this study included males and females 9 to 45 years of age with moderate to severe facial acne vulgaris and no disorders that would preclude the use of tetracycline-class antibiotics. Subjects were treated for a total of 12 weeks and returned to a clinic to be assessed on Weeks 3, 6, 9, and 12 of treatment. Subjects were assessed through lesion counts and Investigator's Global Assessment (“IGA”) scores (as described above in Example 1).

Subjects having facial acne vulgaris with: 20 to 50 inflammatory lesions (papules, pustules, and nodules), up to 100 non-inflammatory lesions (open and closed comedones), no more than two nodules and an IGA score of moderate (3) or severe (4) were included in the study.

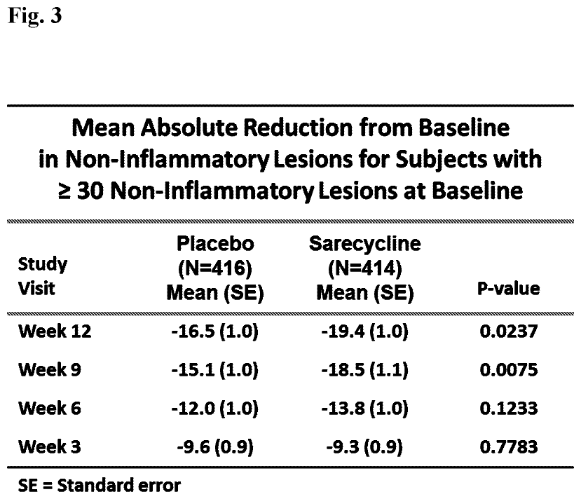

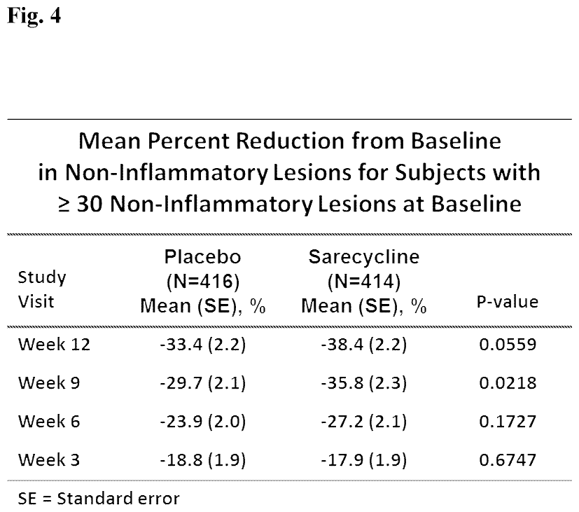

Data was analyzed for a subset of subjects having 30 or more non-inflammatory lesions. The effects of Formula I were evaluated for such subjects in comparison to a placebo. 414 subjects were given 1.5 mg/kg per day of Formula I, and 416 subjects were given a daily dose of a placebo. Each subject was instructed to take the daily dose at the same time each day. FIG. 3 demonstrates the mean absolute reduction in non-inflammatory lesions for participants having 30 or more non-inflammatory lesions on the face at baseline who received daily doses of Formula I compared to the placebo. FIG. 4 demonstrates the mean percent reduction from baseline for subjects having 30 or more non-inflammatory lesions on the face at baseline. Results in these figures were based on a statistical (analysis of covariance) model used to estimate the mean treatment effects of Formula I and placebo, after adjusting for potential effects of investigative site and baseline value. In addition, the statistical superiority of Formula I relative to placebo is tested and p-values provided for each study visit.

### Reference Example

Approximately 3.0 mg/kg per day, approximately 1.5 mg/kg per day, or approximately 0.75 mg/kg per day of the mono hydrochloride crystalline salt of (4S,4aS,5aR,12a5)-4-dimethylamino-3,10,12,12a-tetrahydroxy-7-[(methoxy(methyl)amino)-methyl]-1,11-dioxo-1,4,4a,5,5a,6,11,12a-octahydro-naphthacene-2-carboxylic acid amide (i.e., Formula I) compared to a placebo was administered to subjects including approximately 280 male and female subjects aged 12 to 45 years having moderate to severe facial acne vulgaris. Subjects were randomly assigned in a 1:1:1:1 ratio to one of the said four treatment groups. Subjects were treated for a total of 12 weeks and returned to a clinic to be assessed on Weeks 1, 2, 4, 8, and 12 of treatment. Subjects were assessed through lesion counts and Investigator's Global Assessment (“IGA”) scores.

Subjects having facial acne vulgaris with: 20 to 50 inflammatory lesions, 30 to 100 non-inflammatory lesions, no more than 2 facial nodules and having an Investigator Global Assessment (IGA) at Baseline Visit of “moderate” or “severe” were included in the study.

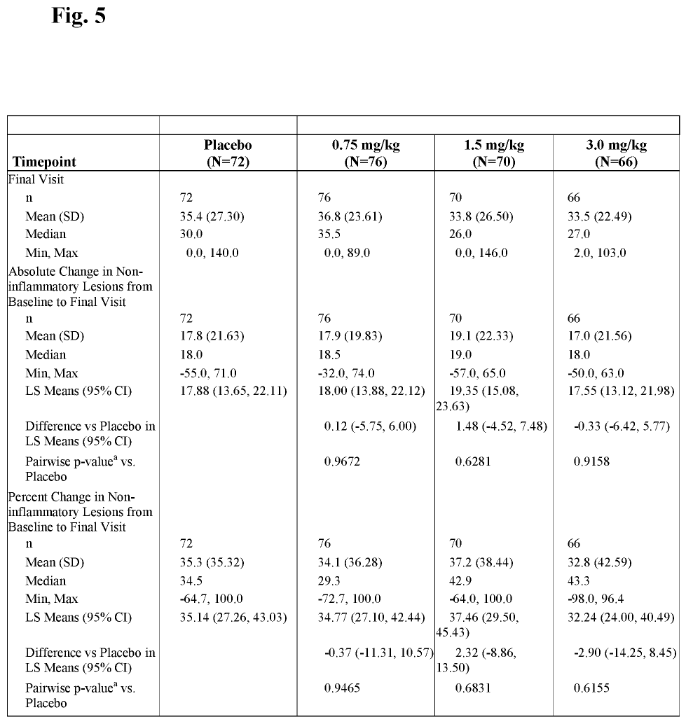

The effects of Formula I were evaluated in comparison to a placebo. In this study, 72 subjects were given a placebo, 76 subjects were given 0.75 mg/kg per day of Formula I, 70 subjects were given 1.5 mg/kg per day of Formula I and 66 subjects were given 3.0 mg/kg per day of Formula I. Each subject was instructed to take the daily dose at the same time each day. FIG. 5 demonstrates the mean absolute and percent change from baseline to final visit.

1.-9. (canceled)

10. A medicament comprising a therapeutically effective amount of (4S,4aS,5aR,12aS)-4-dimethylamino-3,10,12,12a-tetrahydroxy-7-[(methoxy(methyl)amino)-methyl]-1,11-dioxo-1,4,4a,5,5a,6,11,12a-octahydro-naphthacene-2-carboxylic acid amide or a pharmaceutically acceptable salt thereof to treat non-inflammatory lesion and/or symptoms thereof.

11. The medicament of claim 10, wherein the pharmaceutically acceptable salt is selected from the group consisting of a crystalline mono hydrochloride, a crystalline mono mesylate, and a crystalline mono sulfate salt, preferably crystalline mono hydrochloride.

12. The medicament of claim 10, wherein the therapeutically effective amount is administered daily.

13. The medicament of claim 10, wherein the therapeutically effective amount is administered for at least three weeks, at least six weeks, at least nine weeks, or at least twelve weeks.

14. The medicament of claim 10, wherein the therapeutically effective amount is about 1.1 mg/kg/day to about 1.8 mg/kg/day, preferably about 1.5 mg/kg/day.

15. The medicament of claim 10, wherein the therapeutically effective amount is administered as an oral dosage form.

16. The medicament of claim 10, wherein the non-inflammatory lesion is facial acne vulgaris.

17. The medicament of claim 10, wherein the non-inflammatory lesion is an open comedone.

18. The medicament of claim 10, wherein the non-inflammatory lesion is closed comedone.

19. A method of treating a non-inflammatory lesion and/or symptoms thereof comprising administering a therapeutically effective amount of (4S,4aS,5aR,12aS)-4-dimethylamino-3,10,12,12a-tetrahydroxy-7-[(methoxy(methyl)amino)-methyl]-1,11-dioxo-1,4,4a,5,5a,6,11,12a-octahydro-naphthacene-2-carboxylic acid amide or a pharmaceutically acceptable salt thereof to a subject in need thereof.

20. The method of claim 19, wherein the pharmaceutically acceptable salt is selected from the group consisting of a crystalline mono hydrochloride, a crystalline mono mesylate, and a crystalline mono sulfate salt.

21. The method of claims 20, wherein the pharmaceutically acceptable salt is a crystalline mono hydrochloride.

22. The method of claim 19, wherein the therapeutically effective amount is administered daily.

23. The method of claim 19, wherein the therapeutically effective amount is administered for at least three weeks, at least six weeks, at least nine weeks, or at least twelve weeks.

24. The method of claim 19, wherein the therapeutically effective amount is about 1.1 mg/kg/day to about 1.8 mg/kg/day.

25. The method of claim 24, wherein the therapeutically effective amount is about 1.5 mg/kg/day.

26. The method of claim 19, wherein the therapeutically effective amount is administered as an oral dosage form.

27. The method of claim 19, wherein the non-inflammatory lesion is acne vulgaris.

28. The method of claim 27, wherein the acne vulgaris is facial acne vulgaris.

29. The method of claim 19, wherein the non-inflammatory lesion is an open comedone.

30. The method of claim 19, wherein the non-inflammatory lesion is closed comedone.
